(19) United States
(12) Patent Application Publication
(10) Pub. No.: US 2010/0198320 A1
(43) Pub. Date: Aug. 5, 2010

(54) Lateral access blanket

(75) Inventors: Joseph Pierre, Brockton, MA (US); Rachel Starr, Randolph, MA (US); Alan Stec, East Bridgewater, MA (US)

Correspondence Address: LOUIS WOO;LAW OFFICE OF LOUIS WOO, 717 NORTH FAYETTE STREET, ALEXANDRIA, VA, 22314, US

(73) Assignee: Smiths Medical ASD, Inc., Rockland, MA (US)

(21) Appl. No.: 12/320,648

(22) Filed: Jan. 30, 2009

(51) Int. Cl. A61F 7/00

(52) U.S. Cl. 607/107

(57) ABSTRACT

A convective warming blanket is configured to have an upper body portion and two leg portions that extend from a distal end of the upper body portion. The leg portions are separated by a space sufficient to expose a lateral side of a patient covered by the blanket. A flap separable from the leg portion that extends from the mid-section of the upper body portion may be used to securely wrap around the extended arms of the patient, who is lying on his side. Adhesive tapes are provided on the outside edges of the leg portions to secure the blanket to the surface onto which the patient lies. Apertures are appropriately formed at the sheet that comes into contact with the patient at the upper body portion and along the respective inner side edges of the leg portions so that both the upper body and the exposed lower body of the patient are warmed by heated air. The blanket is designed to have mirror image versions usable for exposing the lateral left side or the lateral right side of the patient.

### FIELD OF THE INVENTION

The present invention relates to convective warming blankets, and more particularly relates to a blanket that is designed to allow a clinician or surgeon to gain access to a lateral side of a patient lying on his or her side.

### BACKGROUND OF THE INVENTION

The use of convective warming blankets for warming a patient during surgery is known. Such convective warming blankets include blankets that are used to fully cover the body a patient, or a part of the patient. There are also underbody blankets onto which a patient lies. But currently there are no convective warming blankets in the market that would allow a surgeon to gain access to the lateral side of a patient to perform a procedure, surgical or otherwise such as for example a hip replacement, that requires the patient to lie on his side.

### SUMMARY OF THE PRESENT INVENTION

The convective warming blanket of the present invention is configured to be placed over a patient so that a lateral side of a patient, from the patient's feet to approximately his lower back and stomach, is exposed, to thereby allow a clinician or surgeon to gain access to the patient for those procedures, surgical or otherwise, that require a patient to lie on his side. So that the surgeon may gain access to either the left or the right lateral side of the patient, the blanket of the instant invention may be configured specifically for those instances where the patient is lying either on his right side (a left side lateral access blanket) or on his left side (a right side lateral access blanket).

In particular, the blanket is constructed from two air impermeable sheets that are sealed at their peripheries to provide an inflatable structure. This inflatable structure has an upper body portion that is dimensioned to cover the upper body of the patient. There are extending from the distal end of the upper body portion two longitudinal leg portions that are spatially separated. One of the longitudinal leg portions extends from the left or the right side of the upper body portion, such that the outside periphery of the longitudinal leg portion seamlessly extends from the longitudinal outside edge periphery of the upper body portion. The other longitudinal leg portion extends from the distal end of the upper body portion away from the leftmost and rightmost longitudinal peripheral edges of the upper body portion.

The other longitudinal leg portion extending between the leftmost and the rightmost side edges of the upper body portion and the longitudinal leg portion that has side edges that form a continuous edge with the upper body portion are separated by a space sufficient to accommodate the lower body the patient, as the patient is lying on his or her side.

A flap that is an extension from the other side of the upper body portion away from the longitudinal side edge leg portion extends from the upper body portion to the length of the longitudinal leg portions. The outer side edge of the flap is a continuation of the longitudinal side edge of the upper body portion, while the inner side edge of the flap is removably connected to the longitudinal side edge of the leg portion that extends from approximately the middle of the distal section of the upper body portion, so that the flap can be separated from the longitudinal leg portion. For further discussion, this longitudinal leg portion may be referred to as the mid-section longitudinal leg portion. To effect the removable attachment, spaced cuts are made along the common seal between the flap and the edge of the longitudinal leg portion. Once separated from the mid-section longitudinal leg portion, the flap is foldable about the arm board of the support whereon the patient lies. Adhesive means such as a tape or a sticky surface layer is provided at the proximal or upper edge of the upper body portion so that when the flap is wrapped around the arms of the patient, it may be held in place by being adhesively attached to the adhesive at the proximal end of the upper body portion.

There are additional adhesives, such as for example tapes, provided along the respective edges of the longitudinal leg portions that are not facing each other so that those leg portions may be folded or rolled to adhesively attach to the surface onto which the patient lies, therefore ensuring that the blanket is secured in place relative to the patient. An adhesive tape or layer may also be provided to adhesively secure the blanket to the body of the patient.

To inflate the structure, a temperature regulated fluid such as for example heated air is input to the blanket, by way of at least one input port provided at the upper body portion of the blanket. The heated air is output from the blanket at the sheet or layer of the blanket that is in contact with the patient, by means of a number of apertures formed thereat, for warming both the upper portion of the body as well as the exposed lateral portion of the body of the patient.

The present invention is therefore a convective warming blanket that comprises two air impermeable sheets bonded at their respective peripheral edges to form an inflatable structure having an upper body portion and two longitudinal leg portions each extending from the distal end of the upper body portion. The longitudinal leg portions are separated by a space sufficient to expose a lateral side of a patient, from the feet to at least the hip and/or mid-section of the patient, when the blanket is placed over the patient. One of the longitudinal leg portions has one edge extending from the longitudinal side edge of the upper body portion, whereas the other longitudinal leg portions extends from the distal end of the upper body portion between the two longitudinal side edges (or the rightmost and leftmost edges) of the upper body portion. The two longitudinal leg portions sandwich the lateral side of the patient when the blanket is placed over the patient. There is at least one inlet provided at the upper body portion to enable temperature treated air to be input to the structure for inflating the same. The sheet in contact with the patient has a number of apertures for outputting the temperature treated air to the patient

The present invention also relates to a convective warming blanket that comprises an inflatable structure having an upper body portion, two legs extending from a distal end of the upper body that are separated by a space sufficient to expose a lateral side of patient from his feet to at least his hips when the patient is covered by the blanket. One of the legs extends from the left or right side of the upper body portion, and the other leg extends between the leftmost and the rightmost sides of the upper body portion. A flap extends from the side of the upper body that the one leg does not extend from. At least one inlet is provided to enable temperature treated air to be input to the structure.

The present invention further relates to a convective warming blanket that allows access to a lateral side of a patient which is formed by two air impermeable sheets sealed at their respective peripheries, the blanket comprising: an upper body portion, two legs extending from a distal end of the upper body portion, the legs separated by a space sufficient to expose a lateral side of a patient from his feet to at least his hip when the patient is covered by the blanket, one of the legs extending from the left or the right side of the upper body portion while the other leg extending between the leftmost and the rightmost sides of the upper body portion; at least one inlet to enable heated air to input into and inflate the structure; multiple rows of first directional apertures formed at the sheet in contact with the patient at an area of the upper body portion that covers the arms of the patient for outputting the heated air to at least the arms of the patient; and multiple rows of second directional apertures formed at the sheet in contact with the patient along an area of the upper body portion that covers the upper body of the patient for outputting the heated air to at least the upper body of the patient.

### BRIEF DESCRIPTION OF THE FIGURES

The present invention will become more apparent and the invention itself will be best understood by reference to the following description of the invention taken in conjunction with the following drawings, wherein:

FIG. 1 is a view of the convective blanket of the invention designed to expose the right lateral portion of the body of a patient;

FIG. 2 is a top view of the bottom sheet of the FIG. 1 blanket that comes into contact with a patient;

FIG. 3 is a view of the blanket of the instant invention covering a patient, and showing an exemplar exposed lateral side of a patient; and

FIG. 4 is an interposed view of both sheets of a blanket of the instant invention designed to be placed on a patient to expose the left lateral side of a patient.

### DETAILED DESCRIPTION OF THE INVENTION

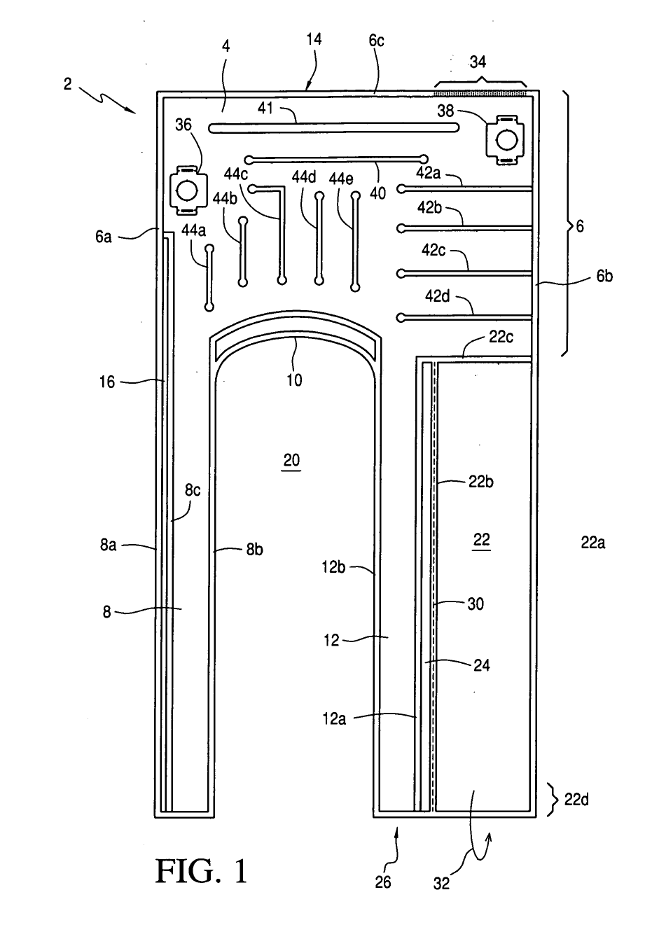

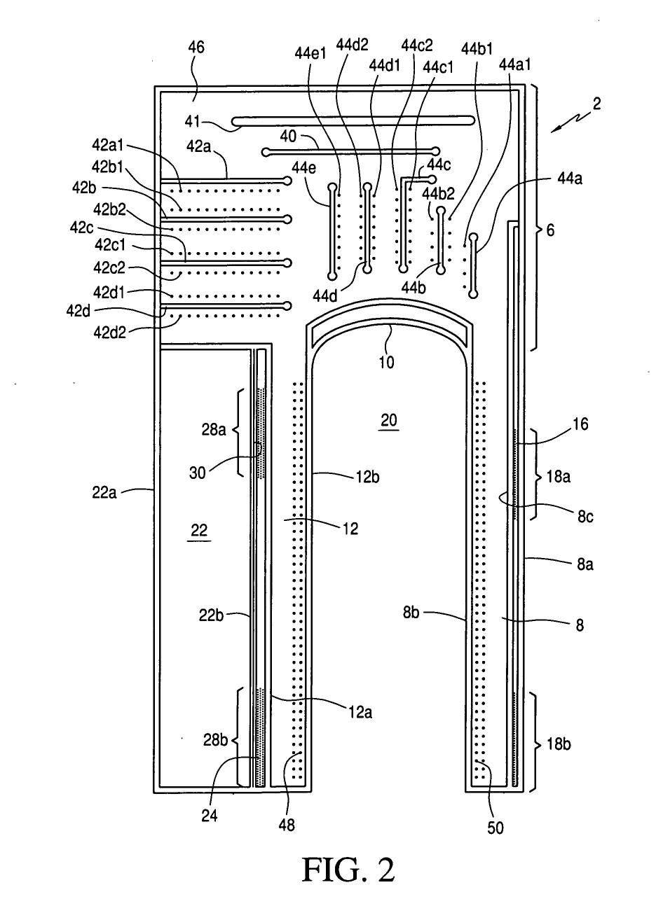

With reference to FIG. 1, an embodiment of the present invention blanket 2 is shown to include an inflatable structure 4 having an upper body portion 6, a first leg portion 8 that extend longitudinally from a distal end 10 of the upper body portion 6, and another leg portion 12 that also extends from the distal portion 10 of the upper body portion 6. Structure 4 is made up of two air impermeable sheets, the bottom sheet shown in FIG. 2, that are bonded at their respective peripheral edges 14.

With reference to the orientation of the blanket as shown in FIG. 1, leg portion 8 is shown to extend from the distal end of upper body portion 6 at its left side, and its left edge 8a is a continuation of the left edge or seal 6a of the upper body portion 6. In other words, a continuous seal runs longitudinally from the proximal edge of the upper body portion 6 to the distal edge of the leg portion 8, per shown on the left side of blanket 2. It should be noted that even thought the discussion herein refers to the seal at the different portions of the blanket with different designated numbers, in actuality, as noted above, the seal runs along the periphery of the blanket, as designated earlier by reference number 14. The designation of the different portions of the seal with different reference numbers is meant to point out with more particularity the different sections of the blankets.

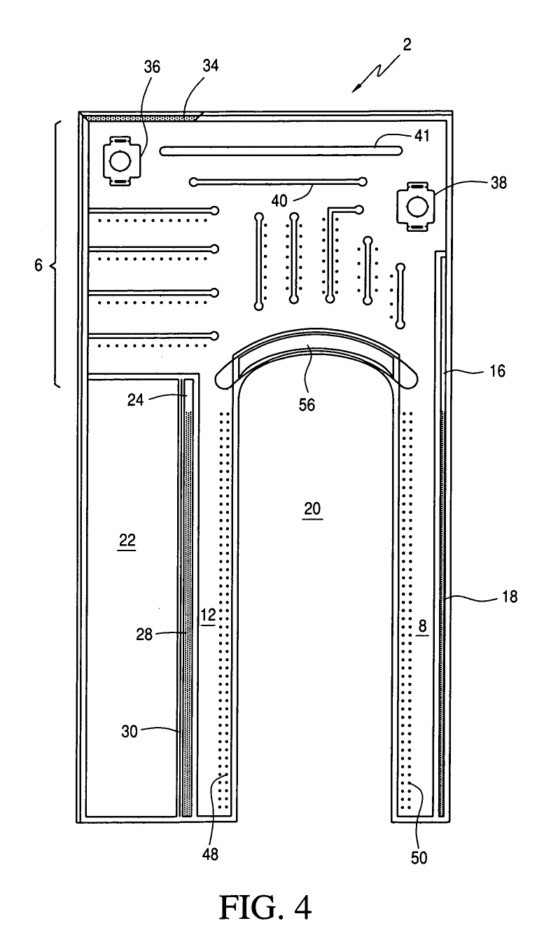

In any event, leg portion 8 is also formed by an inner seal 8b. Within outer seal 8a (6a) there is yet another seal 8c which, together with seal 8a (6a), form a longitudinal non-inflated section 16. Onto section 16 of the sheet that contacts the patient (FIG. 2) there is provided an adhesive material, for example double-sided tapes 18a and 18b. Even though two separate adhesive tapes are shown, in practice, a single adhesive tape may replace tape 18a to 18b. Indeed, such single tape is shown in the embodiment of the blanket illustrated in FIG. 4.

The second leg portion 12 extends longitudinally from the distal end 10 of the upper body portion 6 between the leftmost and rightmost side edges of the blanket. For ease of reference, the leftmost side of the blanket may be referred to by the same designation 6a that references the seal of upper body portion 6 while the rightmost side of the blanket may be referred as 6b, which also designates the right periphery side seal for the upper body portion 6, as viewed from FIG. 1. Leg portion 12 has a right seal or edge 12a and a left seal or edge 12b.

The distal end 10 of the upper body portion 6, the right edge 8b of leg portion 8 and the left edge of 12b of leg portion 12 in combination define a space 20 that extends from approximately the mid-section to the foot or distal end of the blanket.

There is also formed at the blanket a flap 22 that extends from the right side of upper body portion 6 such that its right seal or edge 22a runs continuously from the seal of edge 6b of the upper body portion 6. Flap 22 has a left edge 22b that, together with right edge 12a of leg portion 12 form a non-inflatable space and a common seal. More in particular, edge 12a of leg portion 12 and edge 22b of flap 22 together define a longitudinal space 24 extending substantially from the distal end of the upper body portion 6 to the foot end 26 of the blanket. Inside area 24 on the patient layer (FIG. 2) is at least one, but in this instance two adhesive strips, for example double sided tapes 28a and 28b. Although two different strips are shown, it should be appreciated that a single adhesive strip may extend along the length of area 24, per shown in the embodiment blanket of FIG. 4.

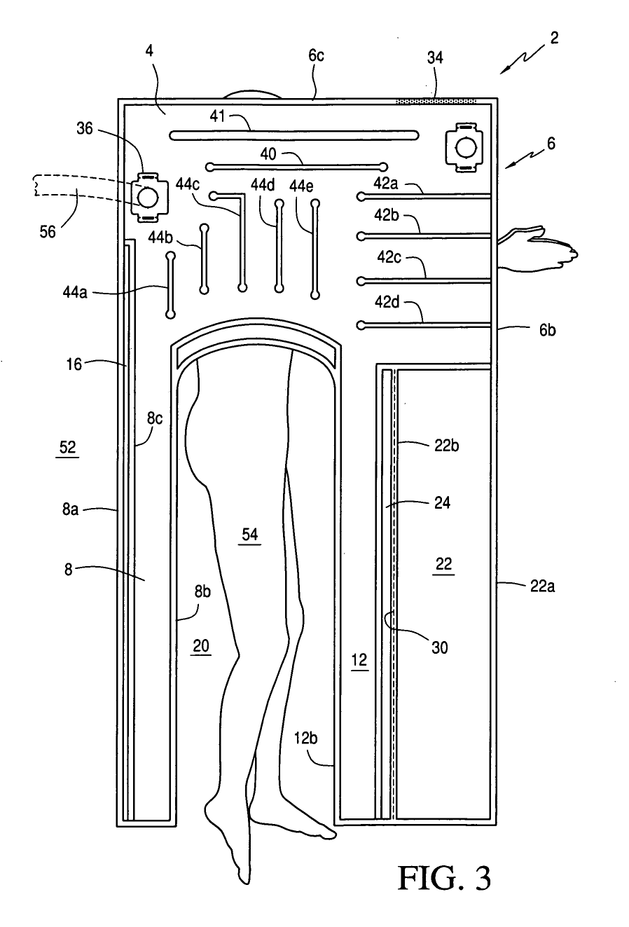

At seal 22b that conceivably could be considered to be the edge that separates flap 22 from leg portion 12, a number of slits 30 running along the length of the seal makes flap 22 removably attached to leg portion 12, so that flap 22 may be separated from leg portion 12 by tearing along the slits 30. Flap 22 is attachedly extending from the distal end of upper body portion 6 by seal 22c, which separates the inflatable upper body portion 6 from flap 22, thereby making flap 22 not inflatable. Once separated from leg portion 12, flap 22 may be folded in a direction into the paper, so that it may wrap around the right side of upper body portion 6, per shown by directional arrow 32. An adhesive such as a double-sided tape 34 may be provided at the proximal end of upper body portion 6, for example at seal 6c, so that when flap 22 is folded per shown by directional arrow 32 (into the page), the distal portion 22d thereof may be adhesively attached to adhesive 34. The purpose of attaching flap 22 to adhesive 34 is to ensure that the arms of the patient are wrapped and secured to the support surface onto which the patient lies, as will be discussed in more detail with reference to FIG. 3.

The convective warming blanket of FIG. 1 is shown to also include an inlet port 36 at the left side of the blanket and an inlet port 38 at the right side of the blanket. Only one inlet port is used at any one time. An air hose is inserted into the opening at the inlet port, and an air warmer, such as for example the Equator Air Warmer being sold by the assignee of the instant invention, is activated to input temperature treated fluid, for example heated air, into the blanket for inflating the same. Inlet ports 36 and 38 each are the same as that disclosed in application Ser. No. 11/401,957 filed by the assignee of the instant application on Apr. 12, 2006. The disclosure of the '957 application is incorporated by reference herein.

With reference to the upper body portion 6, there is shown a number of seals that partition upper body portion 6 into different air flow patterns. These seals include seal 40 and seal 41 that add strength to prevent seal separation located in the top portion of upper body portion 6, seals 42a-42d located at the right portion of upper body portion 6, and seals 44a-44e located substantially in alignment longitudinally with space 20. Note that seals 40 and 42a-42d extend horizontally, with reference to FIG. 1, while seals 44a-44e substantially extend vertically with reference to FIG. 1. The section of upper body portion 6 where seals 44a-44e are located may be referred to as the arm portion of the blanket that extends orthogonally beyond leg portion 12, i.e., at a direction orthogonal to the longitudinal direction of the leg portions 8 and 12.

FIG. 2 is the bottom view of blanket 2 shown from its underside, i.e., the bottom sheet or layer of the two sheet structure. Sheet 46 of blanket 2 in essence is a mirror image of top sheet 4 of blanket 2, but with apertures added thereto for outputting the heated air input to blanket 2 by way of either of the inlet ports 36 and 38.

In particular, on the lower sheet of the blanket which comes into contact with the patient, there are two rows of apertures 48 that extend longitudinally adjacent the edge 12b of leg portion 12 and two rows of apertures 50 located adjacent edge 8b of leg portion 8. With the two sets of two rows of apertures 48 and 50 provided substantially along the respective lengths of the leg portions, when blanket 2 is inflated, heated air is output from those apertures towards space 20, where the lower body of the patient is located, thereby warming the front and back sides of the patient that face the apertures, as well as the exposed lateral side of the patient, as will be discussed with reference to FIG. 3.

There are also respective rows of apertures provided adjacent to and along the horizontal directional seals 42a-42d. These apertures are designated 42a1, 42b1, 42b2, 42c1, 42c2, 42d 1 and 42d2. The respective rows of apertures at seals 42a-42d allow the output of heated air to warm the arms of the patient, as well as the chest area of the patient, as the patient lies sideways. There are further respective rows of apertures for the vertical directional seals 44a-44e. These rows of apertures are designated 44a1, 44b1, 44b2, 44c1, 44c2, 44d1, 44d2 and 44e1 in FIG. 2. The vertical rows of apertures for seals 44a-44e enable the heated air to be output to warm the upper body of the patient being covered by blanket 2.

The positioning of the blanket relative to the patient is illustrated in FIG. 3. For the sake of clarity, the operating table or bed onto which the patient lies is designated as support surface 52. As shown, a patient 54 lying on surface 52 is covered by blanket 2. Shown in dotted lines is an air hose 56 mated to the opening at inlet port 36 for supplying heated air from the air warmer (not shown) into the blanket for inflating the same. As shown, space 20 sandwiched by leg portions 8 and 12 is of a sufficient dimension to expose the lower body of the patient laterally from the patient's feet to at least his legs and buttocks and more likely his lower back and stomach. The patient's arms are extended with the patient's hands positioned outside the blanket for possible medication infusion purposes. Thus, the heated air output from the rows of apertures aligned with seals 42a-42d would warm the arm of the patient and to a certain extent the chest of the patient, as the patient is lying on his left side for the FIG. 3 illustration. At the same time, the heated air output from the vertical rows of apertures that are adjacent to seals 44a-44e would warm the upper body of the patient.

As the patient most likely is intubated, the head of the patient is shown to be substantially covered by the top of upper body portion of the blanket. To secure the patient to the operating table or bed, as was discussed earlier, flap 22 is separated from leg portion 12 by tearing at slits 30, so that flap 22 may be wrapped around the arm of the patient, most likely under the surface whereupon the arms of the patient rest. The distal end of the flap 22 is secured to the adhesive tape 34 at upper seal 6c of upper body portion 6.

With the lateral portion of the lower body of the patient exposed, a surgeon or clinician can readily gain access to selective portions of the lower body of the patient as shown so that a surgical procedure, such as for example a hip replacement that requires the patient to lie on his side, may be performed. At the same time, as heated air is being directed to the exposed lower body of the patient by the respective rows of apertures 48 and 50, the exposed portion of the patient is warmed.

To further secure blanket 2 in place, after flap 22 is separated from leg portion 12 and wrapped around the arms of the patient, the respective outer side edges of leg portions 8 and 12 can be manipulated so that area 16 of leg portion 8 and area 24 of leg portion 12 may be removably secured to support surface 52 by attaching the adhesive layer 18a and 18b of section 16 and 28a and 28b of section 24 to the surface of the table or bed onto which the patient lies. With the leg portions of the blanket adhesively secured to surface 52, the blanket is secured in place. Of course, blanket 2 can readily be removed from surface 52, as the adhesive layers are not permanently attached to surface 52.

To secure the blanket to the patient, an adhesive means, such as for example a tape 56 shown in FIG. 4, may be added for example at the distal end of the upper body portion of the blanket to removably affix the blanket to the patient body.

Since surgical procedures may need to be performed on either lateral side of the patient, the blanket of the instant invention is designed to have two versions. A first is that shown in FIGS. 1-3, which is adapted to be used to expose the patient's right lateral side, i.e., the patient is lying on his left side. For those instances where the patient is to lie on his right side, so that his left lateral side is to be exposed, the instant invention blanket is designed to have the shape as shown in FIG. 4.

As FIG. 4 is a mirror image of the blanket embodiment discussed in FIGS. 1-3, the same number designations apply equally to FIG. 4. One difference in FIG. 4 is that for the sake of convenience, instead of showing both the upper sheet and the lower sheet of the blanket, the apertures of the lower sheet and the upper sheet are superimposed, so that both the apertures and the inlet ports are shown. The difference between the positioning of inlet port 36 and outlet port 38 for the blanket of FIG. 4 and that shown earlier is necessitated by the fact that the FIG. 4 blanket is a mirror image of the blanket shown in FIGS. 1-3. In addition, instead of two separate adhesive sections along leg portions 8 and 12, the adhesive layer now extends continuously along areas 24 and 16, per shown by the exemplar adhesive tapes 28 and 18, respectively. There is also shown adhesive tape 56 at the distal end of upper body portion 6 for securing the blanket to the patient. The operation and the functioning of blanket 2 for the FIG. 4 embodiment are the same those discussed earlier with respect to the blanket embodiment of FIGS. 1-3. Flap 22 is separable from leg portion 12 and wrappable around the arms of the patient and be secured to adhesive 34 at the proximal end of the upper body portion 6; and sections 16 and 24 of leg portions 8 and 12, respectively, may secure the blanket to the surface of the bed or operating table onto which the patient lies and is covered by the blanket. The output of the heated air for warming the patient from the various rows of apertures remain as was discussed.

1. A convective warming blanket comprising: two air impermeable sheets bonded at their respective edges to form an inflatable structure having an upper body portion and two longitudinal leg portions each extending from a distal end of said upper body portion, said longitudinal leg portions separated by a space sufficient to expose a lateral side of a patient from the feet of the patient to at least the legs of the patient when the blanket is placed over the patient, one of the longitudinal leg portions having one edge extending from a side edge of said upper body portion, the other of the longitudinal leg portions extending from the distal end of the upper body portion between the two longitudinal side edges of said upper body portion, the two longitudinal leg portions sandwiching the lateral side of the patient when the blanket is placed over the patient, at least one inlet provided at said upper body portion to enable temperature treated air to be input to said structure for inflating the same, the sheet in contact with the patient having apertures for outputting the temperature treated air to the patient.

2. Blanket of claim 1, wherein said upper body portion comprises an arm portion that extends orthogonally beyond the other longitudinal leg portion for covering the arms of the patient.

3. Blanket of claim 1, further comprising a flap extending from the distal end of said upper body portion, said flap fixedly extending from the distal end of said upper body portion and having one of its longitudinal edge releaseably attached to the longitudinal edge of said other longitudinal leg portion away from said space.

4. Blanket of claim 3, further comprising an adhesive means provided at an upper end of said upper body portion; and
wherein said flap when separated from the longitudinal edge of said other longitudinal leg portion is wrappable about said upper body portion and secure thereto by attaching to the adhesive means.

5. Blanket of claim 1, further comprising adhesive means provided along respective edges of the longitudinal leg portions away from said space, the respective edges of the longitudinal leg portions make adhesive contact with a surface onto which the patient and the blanket are positioned so that the longitudinal leg portions of the blanket may be secured to the surface.

6. Blanket of claim 1, wherein multiple rows of apertures are provided at the sheet in contact with the patient along the direction of the placement of the arms of the patient for outputting temperature treated air to at least the arms of the patient covered by said upper body portion.

7. Blanket of claim 1, further comprising multiple rows of apertures formed longitudinally along said upper body portion of the blanket at the sheet that makes contact with the patient for outputting temperature treated air to at least the upper body of the patient.

8. Blanket of claim 1, wherein there are sets of two longitudinal rows of apertures each provided along a corresponding one of the edges of the longitudinal leg portions that oppose each other for defining said space, temperature treated air output from the two rows of apertures toward said space at the lower body and legs of the patient.

9. Blanket of claim 1, wherein the blanket is configured to cover the patient when the patient is lying on his right side, so that the left lateral side of the patient below his upper body is exposed between the longitudinal leg portions.

10. Blanket of claim 1, wherein the blanket is configured to cover the patient when the patient is lying on his left side, so that the right lateral side of the patient below his upper body is exposed between the longitudinal leg portions.

11. A convective blanket comprising an inflatable structure having an upper body portion, two legs extending from a distal end of said upper body portion, the legs separated by a space sufficient to expose a lateral side of a patient from his feet to at least his legs when the patient is covered by the blanket, one of the legs extending from the left or right side of said upper body portion, the other leg extending between the leftmost and rightmost sides of the upper body portion, a flap extending from the side of said upper body that said one leg does not extend from, and at least one inlet to enable temperature treated air to input to the structure.

12. Blanket of claim 11, wherein said flap and said other leg are separable along a common longitudinal seal, said flap being sealed from said upper body portion and is not inflatable.

13. Blanket of claim 12, further comprising an adhesive means provided at said upper body portion; and
wherein said flap after separated from said other leg is wrappable about said upper body portion and secure thereto by attaching to the adhesive means.

14. Blanket of claim 11, further comprising adhesive means provided along respective opposite longitudinal edges of the two legs for attaching the two legs to a surface onto which the patient and the blanket are positioned.

15. Blanket of claim 11, further comprising multiple rows of apertures formed at the sheet in contact with the patient at an area of said upper body portion that covers the arms of the patient in a direction orthogonal to the legs for outputting the temperature treated air to at least the arms of the patient.

16. Blanket of claim 11, further comprising multiple rows of apertures formed along a longitudinal direction at the sheet in contact with the patient along an area of said upper body portion that covers the upper body of the patient for outputting the temperature treated air to at least the upper body of the patient.

17. Blanket of claim 11, further comprising at least one row of apertures provided longitudinally along an edge of each of the legs facing said space for outputting the temperature treated air to the lower body and legs of the patient.

18. Blanket of claim 11, wherein the blanket is configured to cover the patient and to expose either the left lateral side or the right lateral side of the patient below his upper body.

19. A convective warming blanket allowing access to a lateral side of a patient formed by two air impermeable sheets sealed at their respective peripheries, the blanket comprising:
an upper body portion;
two legs extending from a distal end of said upper body portion, the legs separated by a space sufficient to expose a lateral side of a patient from his feet to at least his legs when the patient is covered by the blanket, one of the legs extending from the left side or the right side of said upper body portion, the other leg extending between the leftmost and rightmost sides of the upper body portion;
at least one inlet to enable heated air to input into and inflate the structure;
multiple rows of first directional apertures formed at the sheet in contact with the patient at an area of said upper body portion that covers the arms of the patient for outputting the heated air to at least the arms of the patient; and
multiple rows of second directional apertures formed at the sheet in contact with the patient along an area of said upper body portion that covers the upper body of the patient for outputting the heated air to at least the upper body of the patient.

20. Blanket of claim 19, wherein each leg comprises at least one row of apertures along a longitudinal edge thereof that faces said space for outputting the heated air to the exposed lateral side of the patient.

21. Blanket of claim 19, further comprising a flap extending from the side of said upper body that said one leg does not extend from, said flap and said other leg being separable along a common longitudinal seal, said flap being sealed from said upper body portion so as not to be inflatable.

22. Blanket of claim 21, further comprising an adhesive means provided at said upper body portion; and
wherein said flap after separated from said other leg is wrappable about said upper body portion and secure thereto by attaching to the adhesive means.

23. Blanket of claim 19, further comprising adhesive means provided along the respective opposite longitudinal edges of the two legs for attaching the two legs to a surface onto which the patient and the blanket are positioned.
